(19) United States
(12) Patent Application Publication
(10) Pub. No.: US 2008/0262453 A1
(43) Pub. Date: Oct. 23, 2008

(54) REMOTE MONITORING DIAPER SYSTEM, KIT AND METHOD OF USING

(76) Inventors: William J. MCGINNIS, Cincinnati, OH (US); Scott A. METRICK, Glenview, IL (US)

Correspondence Address: LADAS & PARRY LLP, 224 SOUTH MICHIGAN AVENUE, SUITE 1600, CHICAGO, IL, 60604, US

(21) Appl. No.: 11/736,808

(22) Filed: Apr. 18, 2007

(51) Int. Cl. A61F 13/15

(52) U.S. Cl. 604/361

(57) ABSTRACT

A remote monitoring system, a kit and an associated method are presented for use in providing a convenient way to alert health care workers that a diaper is in need of being changed. The remote monitoring system includes a remotely placed monitor station in communications with a diaper device. The diaper device has many of the standard diaper components such as having an absorbent pad and absorbent composition interposed between the inner and outer panels. The diaper device also has electronic components that are configured to detect an increase in conductivity across opposing detector electrodes which can then be used to indicate, via an EMF signal, that the diaper device is wet. The electronic components of the remotely placed monitor station are then configured to receive the EMF signal and to configured to alert the health care workers that the diaper device is in need of being changed. The kit includes the electronic components configured to convert a regular diaper into a smart diaper in communications with the monitor station. The method includes the steps of acquiring, activating, allowing, inserting, obtaining, receiving, removing, soiling, transmitting, and wearing.

### FIELD OF THE INVENTION

The present invention relates to hygienic accessories, more particularly to a remote monitoring system, kit and method of using same for indicating a need to change a diaper device when the diaper device is soiled.

### DESCRIPTION OF THE PRIOR ART

Disposable diapers have contributed significantly towards enhancing and maintaining a healthy hygienic environment for the person wearing such a device. This is primarily because the absorbent fabrics, fibers and materials now found in many commercially available disposable diapers are very effective in sequestering waste products away from the epidermis of the wearer. By sequestering the waste away from the epidermis, the conditions for inducing “diaper rash” have been significantly reduced and thus disposable diapers can enhance the hygiene of the person wearing the device.

Although disposable diapers can contribute significantly towards improving the overall health and hygiene of the wearer, it is still prudent and reasonably sound medical advise to remove a disposable diaper once it has been soiled. Because of the remarkable success in absorbing waste products by these commercially available disposable diapers, a potential problem may arise in that the health care worker may become lackadaisical with regards to ignoring a duty of diligently inspecting for soiled conditions within a timely manner of the soiling event. Paradoxically, the success of disposable diapers in being efficient absorbers of waste seems to have given rise to a new need to assure that health care workers remain diligent with regards to replacing disposable diapers when the disposable diapers are soiled.

While disposable diaper devices fulfill their respective, particular objectives and requirements, the aforementioned disposable diapers do not describe a remote monitoring system, a kit and an associated method are presented for use in providing a convenient way to alert health care workers that a diaper is in need of being changed. The remote monitoring system includes a remotely placed monitor station in communications with a diaper device. The diaper device has many of the standard diaper components such as having an absorbent pad and absorbent composition interposed between the inner and outer panels. The diaper device also has electronic components that are configured to detect an increase in conductivity across opposing detector electrodes which can then be used to indicate, via an EMF signal, that the diaper device is wet. The electronic components of the remotely placed monitor station are then configured to receive the EMF and to alert the health care workers that the diaper device is in need of being changed. Nor do the aforementioned disposable diapers describe a kit having the electronic components configured to convert a regular diaper into a smart diaper in communications with the monitor station. Further none of the aforementioned disposable diaper devices disclose a method having the steps of acquiring, activating, allowing, inserting, obtaining, receiving, removing, soiling, transmitting, and wearing.

Therefore, a need exists for a new and improved remote monitoring system having a remotely placed monitor station in communications with a diaper device, in which the diaper device has many of the standard diaper components such as having an absorbent pad and absorbent composition interposed between the inner and outer panels. Further, the diaper device also has electronic components that are configured to detect an increase in conductivity across opposing detector electrodes which can then be used to indicate, via an EMF signal, that the diaper device is wet. The electronic components of the remotely placed monitor station are then configured to receive the EMF and to alert the health care workers that the diaper device is in need of being changed. In this respect, the remote monitoring system, the kit and associated method of using embodiments of the present invention substantially departs from the conventional concepts and designs of the prior art, and in doing so provides a remote monitoring system, a kit and an associated method of using forof providing a convenient means for alerting health care workers that a diaper is in need of being changed.

### SUMMARY OF THE INVENTION

The present system, kit and assocaited method of using, according to the principles of the present invention, overcomes a number of the shortcomings of the prior art by providing a novel remote monitoring system, kit and associated method for use in providing a convenient way to alert health care workers that a diaper is in need of being changed. The remote monitoring system includes a remotely placed monitor station in communications with a diaper device. The diaper device has many of the standard diaper components such as having an absorbent pad and absorbent composition interposed between the inner and outer panels. The diaper device also has electronic components that are configured to detect an increase in conductivity across opposing detector electrodes which can then be used to indicate, via an EMF signal, that the diaper device is wet. The electronic components of the remotely placed monitor station are then configured to receive the EMF and to alert the health care workers that the diaper device is in need of being changed. The kit includes the electronic components which can be used to convert a regular commercially available disposable diaper into the smart diaper device of the remote monitoring system. The method includes the steps of acquiring, activating, allowing, inserting, obtaining, receiving, removing, soiling, transmitting, and wearing.

In view of the foregoing disadvantages inherent in the known type diaper devices, systems, kits, and methods now present in the prior art, the present invention provides an improved remote monitoring system, kit and associated method for use in providing a convenient way to alert health care workers that a diaper is in need of being changed. The remote monitoring system, kit and associated method will be described subsequently in great detail, is to provide a new and improved remote monitoring system, kit and associated method which are not anticipated, rendered obvious, suggested, or even implied by the prior art, either alone or in any combination thereof.

To attain this, the present invention of the remote monitoring system essentially comprises a remote monitor station in communications with a diaper device. The diaper device has many of the standard diaper components such as having an absorbent pad and absorbent composition interposed between the inner and outer panels. The diaper device also has electronic components that are configured to detect an increase in conductivity across opposing detector electrodes which can then be used to indicate, via an EMF signal, that the diaper device is wet. The electronic components of the remotely placed monitor station are then configured to receive the EMF and to alert the health care workers that the diaper device is in need of being changed.

There has thus been outlined, rather broadly, the more important features of the invention in order that the detailed description thereof that follows may be better understood, and in order that the present contribution of the art may be better appreciated.

The invention may also include an optional adhesive tab, an optional elastic member, an optional thermocouple, and optional fragrant agent, and an optional software packet.

Numerous objects, features and advantages of the present invention will be readily apparent to those of ordinary skill in the art upon reading of the following detailed description of presently preferred, but nonetheless illustrative, embodiments of the present invention when taken in conjunction with the accompany drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an aspect of the present invention to provide a new and improved remote monitoring system, kit and associated method that exhibits many of the advantages of the prior diaper devices, kits and associated methods while minimizing a number of their respective disadvantages.

It is another aspect of the present invention to provide a new and improved remote monitoring system and associated kit that may be easily and efficiently manufactured and marketed.

An even further aspect of the present invention is to provide a new and improved remote monitoring system and associated kit that has a low cost of manufacture with regard to both materials and labor, and which accordingly is then susceptible of low prices of sale to the consuming public, thereby making the remote monitoring system and associated kit economically available to the buying public.

Still another aspect of the present invention is to provide a remote monitoring system and associated kit which provides in the apparatuses and methods of the prior art some of the advantages thererof, while simultaneously overcoming some of the disadvantages normally associated therewith.

Even still another aspect of the present invention is to provide a remote monitoring system having a remotely placed monitor station in communications with a diaper device. The diaper device has many of the standard diaper components such as having an absorbent pad and absorbent composition interposed between the inner and outer panels. The diaper device also has electronic components that are configured to detect an increase in conductivity across opposing detector electrodes which can then be used to indicate, via an EMF signal, that the diaper device is wet. The electronic components of the remotely placed monitor station are then configured to receive the EMF and to alert the health care workers that the diaper device is in need of being changed.

Still another aspect of the present invention is to provide a kit includes electronic components configured to convert a regular diaper into a smart diaper for communications with the monitor station.

Lastly, it is an aspect of the present invention to provide a new and improved method of using comprising the steps of acquiring, activating, allowing, inserting, obtaining, receiving, removing, soiling, transmitting, and wearing.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be had to the accompanying drawings and description matter in which there are illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a perspective view of an embodiment of the remote monitoring system constructed in accordance with the principles of the present invention;

FIG. 2 is a close up perspective view of a diaper device of the remote monitoring system of present invention;

FIG. 3 is a perspective view of a regular disposable diaper with a kit of the present invention embedded (not shown) within the regular disposable diaper;

FIG. 4 is a cross sectional view of a regular disposable diaper with a kit of the present invention embedded (shown) within the regular disposable diaper;

FIGS. 5A and 5B are top and side views, respectively, of a kit of the present invention; and

FIG. 6 is a perspective view of a monitor station of the remote monitoring system of the present invention.

The same reference numerals refer to the same parts throughout the various figures.

### DETAILED DESCRIPTION OF THE PREFERRED EMBODIMENT

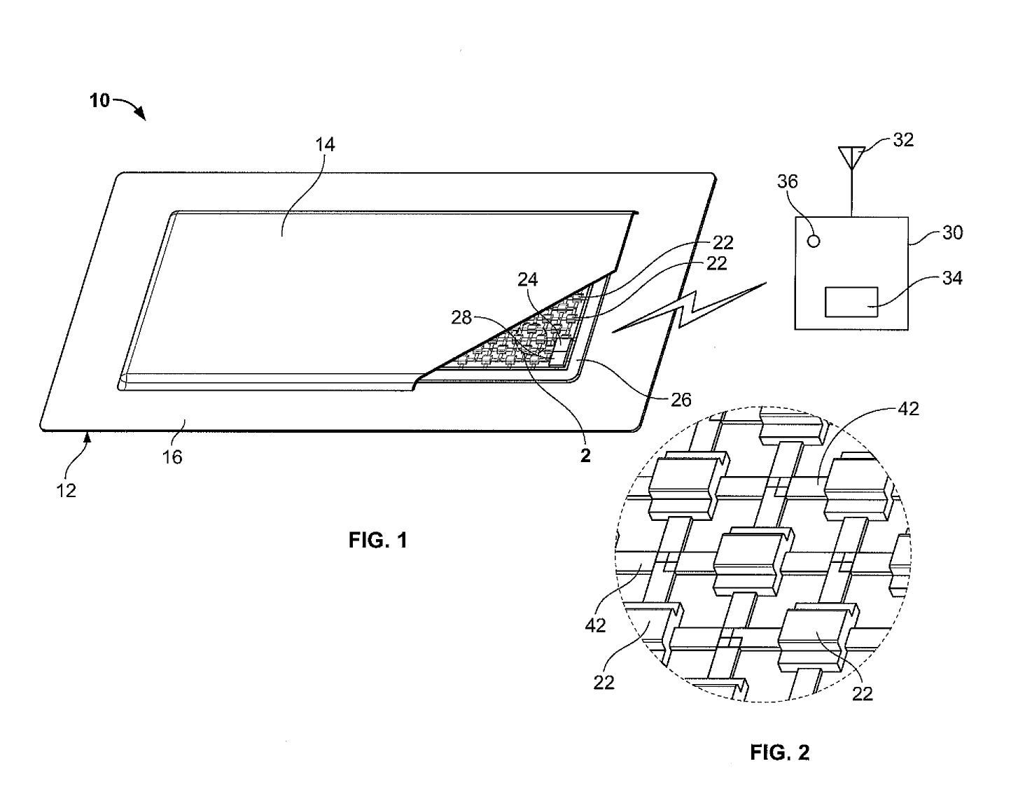

Referring now to the drawings, and in particular FIGS. 1 to 6 thereof, One embodiment of a remote monitoring system 10 for use in indicating a need to change a diaper device 12 when soiled comprises: the diaper device 12 and a monitor station 30. The diaper device 12 comprises: an inner panel 14, an outer panel 1, an absorbent pad 18, an absorbent composition 20, at least one pair of detector electrodes 22, a detector circuit 24, a transmitter 26, and a power supply 28. The monitor station 30 comprises an antenna 32, a receiver circuit 34, and a beacon 36. The inner panel 14 is configured to be liquid pervious. The outer panel 16 is attached to the inner panel 14 in which the outer panel 16 is configured to be substantially liquid impervious. The absorbent pad 18 is interposed between the inner and outer panels (14 and 16, respectively) in which the absorbent pad 18 in fluid communications with the inner panel 14. The absorbent composition 20 is distributed throughout and onto the pad 18. The detector electrodes 22 are interposed between the inner and outer panels (14 and 16, respectively). The detector circuit 24 is operatively coupled to the detector electrodes 22. The transmitter 26 is operatively coupled to the detector circuit 24, in which the transmitter 26 is configured to transmit an EMF signal. The power supply 28 is coupled to the detector electrodes 22, to the circuit and to the transmitter 26. The antenna 32 of the monitor station 30 is configured to be responsive to the transmitted EMF signal. The receiver circuit 34 of the monitor station 30 is operatively coupled to the antenna 32. The beacon 36 of the monitor station 30 is operatively coupled to the receiver circuit 34.

The detector electrodes 22 of the remote monitoring system 10 may be positioned anywhere along the diaper device 12. One preferred embodiment is that the detector electrodes 22 are attached to the outer panel 16. Another preferred embodiment is that the detector electrodes 22 are attached to the absorbent pad 18.

The detector circuit 24 of the remote monitoring system 10 may be positioned anywhere along the diaper device 12. One preferred embodiment is that the detector circuit 24 is attached to the outer panel 16. Another preferred embodiment is that the detector circuit 24 is attached to the absorbent pad 18.

The power supply 28 of the remote monitoring system 10 may be positioned anywhere along the diaper device 12. One preferred embodiment is that the power supply 28 is attached to the outer panel 16. Another preferred embodiment is that the power supply 28 is attached to the absorbent pad 18. The power supply 28 may be any commercially available supply 28 such as those selected from the group consisting of a battery and a high capacity capacitor. When the power supply 28 is in the form of a battery it may be selected from the group consisting of a zinc-carbon battery, zinc-chloride battery, an alkaline/manganese battery, a silver-oxide battery, a lithium battery, a mercury battery, and a water-activated battery. Furthermore, when the power supply 28 is in the form of a water-activated battery the anode may be selected from a magnesium anode, and a magnesium-zinc alloy, and the cathode maybe selected from the group consisting of silver chloride, cuprous chloride, cuprous bromide, cuprous iodide, and cuprous thiocyanate, copper sulfate, and manganese dioxide.

The transmitter 26 of the remote monitoring system 10 maybe positioned anywhere along the diaper device 12. One preferred embodiment is that the transmitter 26 is attached to the absorbent pad 18. Another preferred embodiment is that the transmitter 26 is attached to the outer panel 16.

The beacon 36 of the remote monitoring system 10 may be any commercially available beacon 36 such as those selected from the group consisting of an computer monitor beacon 36, electromagnetic speaker beacon 36, a piezoelectric speaker beacon 36, a light emitting diode beacon 36, a liquid crystal diode beacon 36, and an incandescent lamp beacon 36.

The absorbent pad 18 of the diaper device 12 of the system 10 may be made of any commercially available material such as those materials selected from the group consisting of fluffed cellulose fibers, textile fibers, web of polymeric fibers, wood pulp fibers, polyester, polypropylene, polyurethane, cellulose sponge, and hydrophilic synthetic sponge.

The outer panel 16 of the diaper device 12 of the system 10 may be made of any commercially available material such as those selected from the group consisting of polypropylene polyamide, polyester, polyethylene, ethylene-vinyl acetate, polyurethane, polyolefin blends, co-polyesters, block copolymers and admixtures thereof.

The absorbent composition 20 of the diaper device 12 of the system 10 may be made of any commercially available material such as those superabsorbent polymers selected from the group consisting of polyacrylate polymers, starch graft copolymers, cellulose graft copolymers, cross-linked carboxymethylcellulose derivatives, and admixtures thereof.

An optional software packet 38 may be added to the system 10 in which the optional of software packet 38 is configured to drive the antenna 32, the receiver circuit 34 and the beacon 36 when the receiver circuit 34 is operatively interfaced to a personal computer 40.

An optional networking 42 of interconnected detector electrodes 22 of the system 10 may be added in which each detector electrode 22 in the networking 42 is operatively coupled to the circuit.

An optional adhesive tab 44 may be added to the system 10 in which the at least one adhesive tab 44 is attached to the outer panel 16.

An optional elastic member 46 may be added to the system 10 in which the elastic member 46 is bonded under tension to the outer panel 16.

An optional thermocouple 48 may be added to the system 10 in which the thermocouple 48 maybe interposed between the inner and outer panels (14 and 16, respectively) wherein the thermocouple 48 operatively coupled to the circuit.

An optional fragrant agent 50 may be added to the system 10 in which the fragrant agent 50 may be interposed between the inner and outer panels (14 and 16, respectively). The optional fragrant agent 50 may be selected from the group consisting of apple essence, balsam essence, benzoin resin, blueberry essence, cassia oil, cedar oil, cinnamon essence, clove oil, coriander essence, eucalyptus essence, fresh peach essence, jasmine essence, labdanum resin, lavender essence, lemon essence, lemon oil, musk essence, nutmeg essence, olibanum resinoid, orange oil, patchouli essence, Peru balsam, pine oil, raspberry essence, rose extract sandalwood oil, spearmint essence, styrax, vanilla essence, wintergreen essence, 4-acetyl-6-tert-butyl-1,1-dimethylindane, 5-acetyl-3-isopropyl-1,1,2,6-tetramethylindane, 6-acetyl-1,1,2,3,3,5-hexamethylindane, 7-acetyl-1,1,3,4,4,6-hexamethyltetralin, 7-acetyl-1,2,3,4,5,6,7,8-octahydro-1,1,6,7-tetramethylnaphthalene, ambroxane, amylcinnamaldehyde, anisaldehyde, benzophenone, benzyl acetate, benzyl salicylate, caryophyllene alcohol, cedrol, cedryl acetate, condensation products of hydroxycitronellal and methyl anthranilate, condensation products of hydroxycitronellal and indole, condensation products of phenylacetaldehyde and indole, coumarin, cyclopentadecanolide, γ-decalactone, 2-(1,1-dimethylethyl)cyclo-hexanol acetate, dodecahydro-3a,6,6,9a-tetramethylnaphtho[2,1b]furan, 1-dodecanal, 2-ethyl-4-(2,2,3-trimethyl-3-cyclopenten-1-yl)-2-buten-1-ol, ethylvanillin, formyltricyclodecane, geraniol, heliotropin, hexylcinnamaldehyde, 4-(4-hydroxy-4-methylpentyl)-3-cyclohexene-1-carboxaldehyde, 7-hydroxy-3,7-dimethyloctanal, 16-hydroxy-9-hexadecenoic acid lactone, 1,3,4,6,7,8-hexahydro-4,6,6,7,8,8-hexamethylcyclopenta-γ-2-benzopyran, hydroxyphenylbutanone, α-ionone, β-ionone, γ-ionone, isohexenylcyclohexylcarboxaldehyde, α-isomethylionone, linalyl acetate, 2-methyl-2-(isopropylphenyl)propionaldehyde, 2-methyl-2-(para-isopropylphenyl)propionaldehyde, 2-methyl-3-(para-tert-butylphenyl)propionaldehydc, 2-methyl-3-(tert-butylphenyl)propionaldehyde, methyl1,6,10-trimethyl-2,5,9-cyclododecatrien-1-yl ketone, methylcedrylone, methyl dihydrojasmonate, methyl β-naphthyl ketone, methyl γ-naphthyl ketone, γ-naphthol methyl ether, nerol, para-tert-butylcyclohexyl acetate, tert-butylcyclohexyl acetate, tricyclodecenyl propionate, tricyclodecenyl acetate, 5-(2,2,3-trimethylcyclopent-3-enyl)-3-methylpentan-2-ol, tricyclodecenyl acetate, tricyclodecenyl propionates, phenylethyl alcohol, terpineol, linalool, and 10-undecen-1-al.

One preferred embodiment of a kit 52 for use in converting a regular disposable diaper 54 into a smart diaper device 12 for use in communication with a remote monitor station 30, said kit 52 comprising: at least one pair of detector electrodes 22, a detector circuit 24, a transmitter 26, and a power supply 28. The detector circuit 24 is configured to be operatively coupled to the detector electrodes 22 of the kit 52. The transmitter 26 of the kit 52 is configured to be operatively coupled to the detector circuit 24 of the kit 52 in which the transmitter 26 is also configured to transmit an EMF signal. The power supply 28 of the kit 52 is configured to be coupled to the detector electrodes 22 of the kit 52.

An optional monitor station 30 may be added to the kit 52 in which the monitor station 30 comprises an antenna 32, a receiver circuit 34, and a beacon 36. The antenna 32 of the optional monitor station 30 is configured to be responsive to the transmitted EMF signal. The receiver circuit 34 of the optional monitor station 30 of the kit 52 is configured to be operatively coupled to the antenna 32. The beacon 36 of the optional monitor station 30 of the kit 52 is configured to be operatively coupled to the receiver circuit 34.

An optional software packet 38 may be added to the optional monitor station 30 of the kit 52 in which the software packet is configured to drive the antenna 32, the receiver circuit 34 and the beacon 36 when the receiver circuit 34 is operatively interfaced to a personal computer 40.

An optional thermocouple 48 may be added to the kit 52 in which the thermocouple 48 is configured to be operatively coupled to the detector circuit 24.

One preferred embodiment of a method of using a kit 52 to convert a regular disposable diaper 54 into a smart diaper device 12 for use in communications with a remote monitor station 30 comprises the steps of acquiring, activating, allowing, inserting, obtaining, receiving, removing, soiling, transmitting, and wearing. The obtaining step comprises obtaining the kit 52 comprising: at least one pair of detector electrodes 22; a detector circuit 24 operatively coupled to the detector electrodes 22; a transmitter 26 operatively coupled to the detector circuit 24, the transmitter 26 configured to transmit an EMF signal; a thermocouple 48 operatively coupled to the detector circuit 24; a power supply coupled to the detector electrodes 22, the detector circuit 24, the thermocouple 48 and the transmitter 26; and the monitor station 30 comprising: an antenna 32 configured to be responsive to the transmitted EMF signal; a receiver circuit 34 operatively coupled to the antenna 32; and a beacon 36 operatively coupled to the receiver circuit 34. The acquiring step comprises acquiring an unsoiled regular disposable diaper 54. The inserting step comprises inserting the detector electrodes 22, the detector circuit 24, the transmitter 26, the thermocouple 48 and the power supply 28 of the kit 52 into the acquired regular disposable diaper 54, wherein the steps of inserting results in converting the regular disposable diaper 54 into a smart diaper device 12. The wearing step comprises wearing the smart diaper device 12. The soiling step comprises soiling the smart diaper device 12. The allowing step comprises allowing the detector circuit 24 of the kit 52 to sense an increase in conductivity between the detector electrodes 22 of the kit 52 when the smart diaper device 12 is soiled. The transmitting step comprises transmitting the EMF signal from the transmitter 26 of the kit 52 when the detector circuit 24 of the kit 52 senses the increase in conductivity between the detector electrodes 22 of the kit 52. The receiving step comprises receiving the transmitted EMF signal with the antenna 32 coupled to the receiver circuit 34 of the monitor station 30. The activating step comprises activating the beacon 36 of the monitor station 30 in response to receiving the transmitted EMF signal. The removing step comprises removing the soiled smart diaper device 12 in response to the activated beacon 36.

Referring now to FIG. 1 which depicts a perspective view of an embodiment of the remote monitoring system 10 showing a diaper device 12 and a monitor station 30. The diaper device 12 is shown having an inner panel 14, an outer panel 16, a networking 42 of detector electrodes 22 interposed between the inner and outer panels (14 and 16, respectively), a detector circuit 24, a transmitter 26; and a power supply 28. The monitor station 30 is shown having an antenna 32, a receiver circuit 34, and a beacon 36.

Referring now to FIG. 2 which depicts a close up perspective view of a part of the diaper device 12 of the remote monitoring system 10 showing the networking 42 of detector electrodes 22.

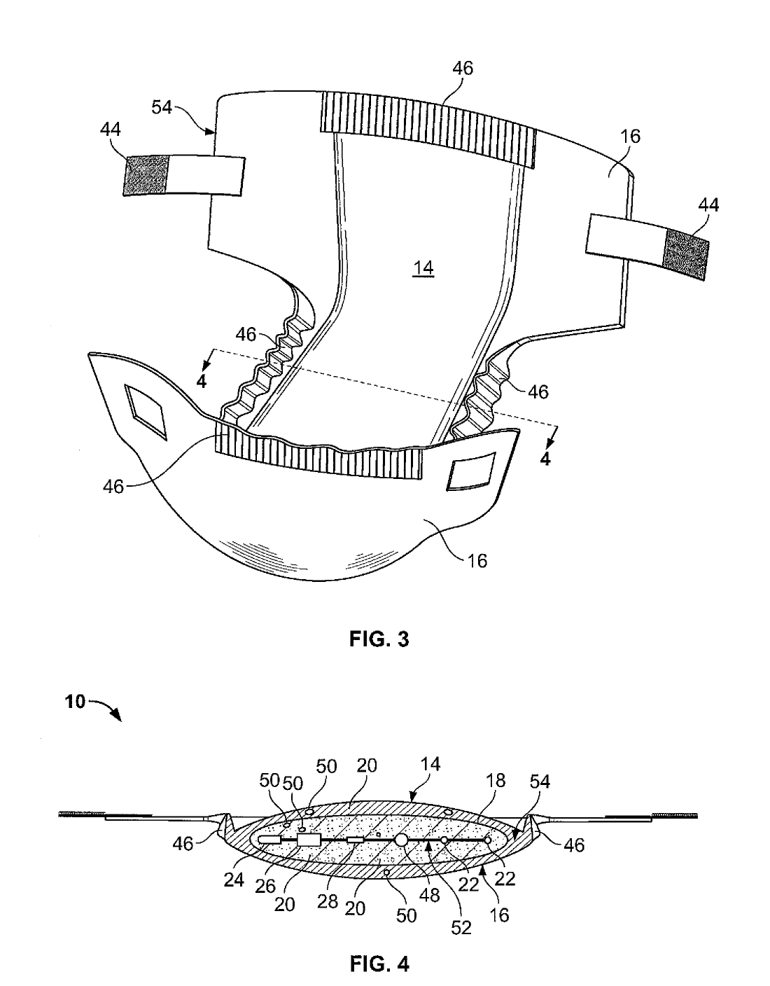

Referring now to FIG. 3, a regular disposable diaper 54 is depicted having a kit 52 (not shown) of the present invention embedded into the regular disposable diaper 54. The disposable diaper 54 is shown having an inner panel 14, an outer panel 16, a plurality of elastic members 46 and a plurality of adhesive tabs 44.

Referring now to FIG. 4, a cross sectional view of a regular disposable diaper with a kit 52 embedded into the regular disposable diaper 54 is depicted. The regular disposable diaper is depicted having an inner panel 14, an outer panel 16, an absorbent pad 18, an absorbent composition 20 distributed onto the pad 18; two elastic members 46 and a fragrant agent 50. The kit 52 is shown embedded within the pad 18 of the regular disposable diaper. The kit 52 is also shown to have a detector electrodes 22, a detector circuit 24, a transmitter 26, a power supply 28, and a thermocouple 48.

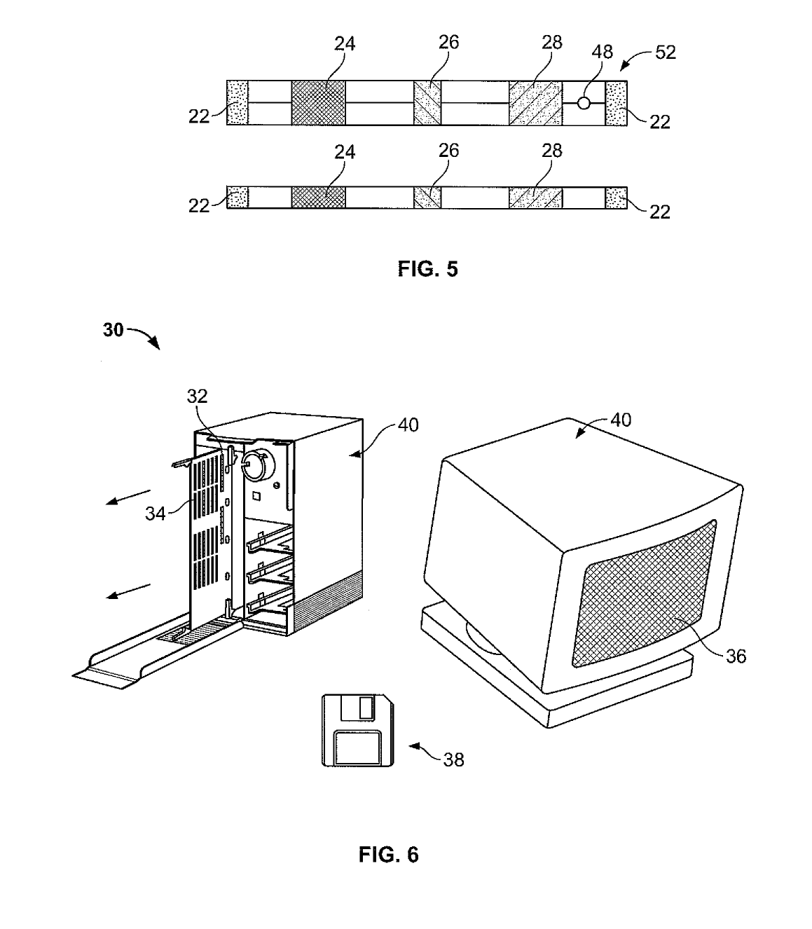

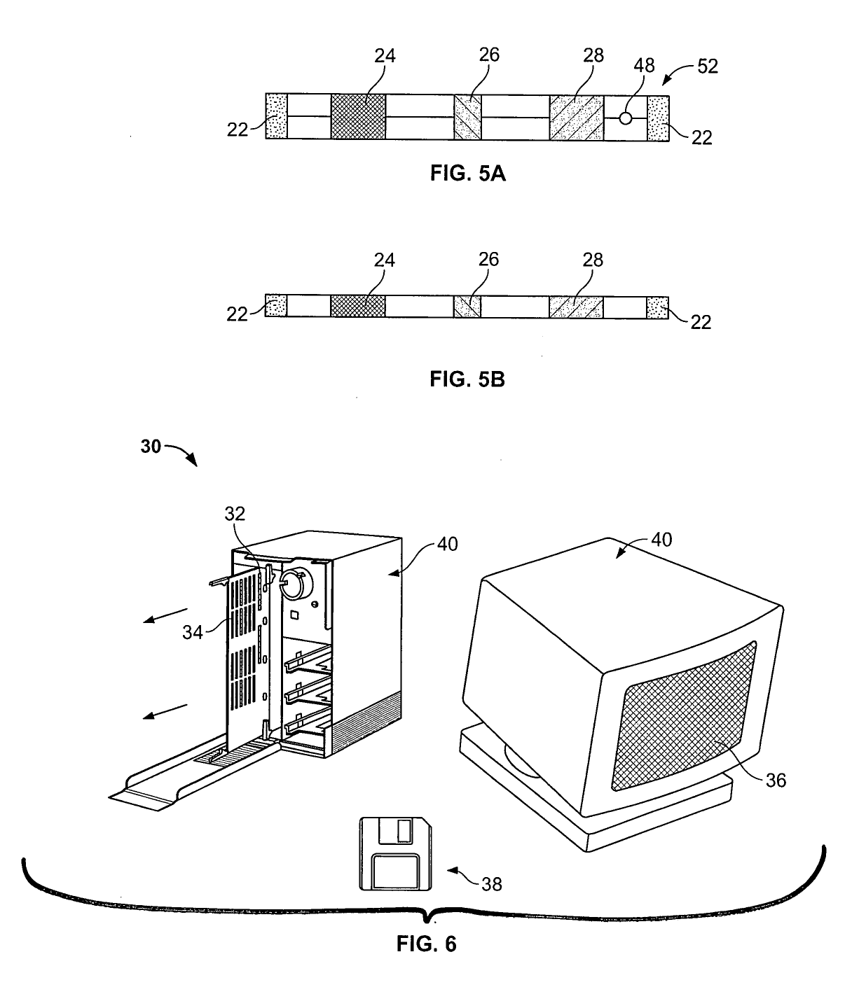

Referring now to FIGS. 5A and 5B which depict respective top and side views of a kit 52 in which the kit 52 is shown having a detector electrodes 22, a detector circuit 24, a transmitter 26, a power supply 28, and a thermocouple 48.

Referring now to FIG. 6 which depicts a perspective view of a monitor station 30 of the present invention. The monitor station 30 is shown having a an antenna 32 and a receiver circuit 34 configured to be operatively coupled to a personal computer 40 so that the beacon 36 (i.e., the personal computer 40 monitor) can be conveniently viewed by health care workers at a remote work area. Also shown is a software packet 38 which is configured to drive the antenna 32, receiver circuit 34 and beacon 36 when the receiver circuit 34 is operatively interfaced to the personal computer 40.

As to the manner of usage and operation of the present invention, the same should be apparent from the above description. Accordingly, no further discussion relating to the manner of usage and operation will be provided.

While a preferred embodiment of the remote monitoring system, kit and method has been described in detail, it should be apparent that modifications and variations thereto are possible, all of which fall within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Throughout this specification, unless the context requires otherwise, the word “comprise” or variations such as “comprises” or “comprising” or the term “includes” or variations, thereof, or the term “having” or variations, thereof will be understood to imply the inclusion of a stated element or integer or group of elements or integers but not the exclusion of any other element or integer or group of elements or integers. In this regard, in construing the claim scope, an embodiment where one or more features is added to any of the claims is to be regarded as within the scope of the invention given that the essential features of the invention as claimed are included in such an embodiment.

Those skilled in the art will appreciate that the invention described herein is susceptible to variations and modifications other than those specifically described. It is to be understood that the invention includes all such variations and modification which fall within its spirit and scope. The invention also includes all of the steps, features, compositions and compounds referred to or indicated in this specification, individually or collectively, and any and all combinations of any two or more of said steps or features.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

We claim:

1. A remote monitoring system for use in indicating a need to change a diaper device when soiled, said system comprising:
the diaper device comprising:
an inner panel being liquid pervious;
an outer panel attached to the inner panel, the outer panel being substantially liquid impervious;
an absorbent pad interposed between the inner and outer panels, the absorbent pad in fluid communications with the inner panel;
an absorbent composition distributed onto the pad;
at least one pair of detector electrodes interposed between the inner and outer panels;
a detector circuit operatively coupled to the detector electrodes;
a transmitter operatively coupled to the detector circuit, the transmitter configured to transmit an EMF signal; and
a power supply coupled to the detector electrodes, the circuit and the transmitter; and
a monitor station comprising:
an antenna configured to be responsive to the transmitted EMF signal;
a receiver circuit operatively coupled to the antenna; and
a beacon operatively coupled to the receiver circuit.

2. The system of claim 1 further comprising a software packet configured to drive the antenna, receiver circuit and beacon when the receiver circuit is operatively interfaced to a personal computer.

3. The system of claim 1 further comprising a networking of interconnected detector electrodes wherein each detector electrode being operatively coupled to the circuit.

4. The system of claim 1 further comprising at least one adhesive tab attached to the outer panel.

5. The system of claim 1 further comprising at least one elastic member bonded under tension to the outer panel.

6. The system of claim 1 further comprising a thermocouple interposed between the inner and outer panels, the thermocouple operatively coupled to the circuit.

7. The system of claim 1 further comprising a fragrant agent interposed between the inner and outer panels, the fragrant agent is selected from the group consisting of apple essence, balsam essence, benzoin resin, blueberry essence, cassia oil, cedar oil, cinnamon essence, clove oil, coriander essence, eucalyptus essence, fresh peach essence, jasmine essence, labdanum resin, lavender essence, lemon essence, lemon oil, musk essence, nutmeg essence, olibanum resinoid, orange oil, patchouli essence, Peru balsam, pine oil, raspberry essence, rose extract, sandalwood oil, spearmint essence, styrax, vanilla essence, wintergreen essence, 4-acetyl-6-tert-butyl-1,1-dimethylindane, 5-acetyl-3-isopropyl-1,1,2,6-tetramethylindane, 6-acetyl-1,1,2,3,3,5-hexamethylindane, 7-acetyl-1,1,3,4,4,6-hexarnethyltetralin, 7-acetyl-1,2,3,4,5,6,7,8-octahydro-1,1,6,7-tetramethylnaphthalene, ambroxane, amylcinnamaldehyde, anisaldehyde, benzophenone, benzyl acetate, benzyl salicylate, caryophyllene alcohol, cedrol, cedryl acetate, condensation products of hydroxycitronellal and methyl anthranilate, condensation products of hydroxycitronellal and indole, condensation products of phenylacetaldehyde and indole, coumarin, cyclopentadecanolide, γ-decalactone, 2-(1,1-dimethylethyl)cyclo-hexanol acetate, dodecahydro-3a,6,6,9a-tetramethylnaphtho[2,1b]furan, 1-dodecanal, 2-ethyl-4-(2,2,3-trimethyl-3-cyclopenten-1-yl)-2-buten-1-ol, ethylvanillin, formyltricyclodecane, geraniol, heliotropin, hexylcinnamaldehyde, 4-(4-hydroxy-4-methylpentyl)-3-cyclohexene-1-carboxaldehyde, 7-hydroxy-3,7-dimethyloctanal, 16-hydroxy-9-hexadecenoic acid lactone, 1,3,4,6,7,8-hexahydro-4,6,6,7,8,8-hexamethylcyclopenta-γ-2-benzopyran, hydroxyphenylbutanone, α-ionone, β-ionone, γ-ionone, isohexenylcyclohexylcarboxaldehyde, α-isomethylionone, linalyl acetate, 2-methyl-2-(isopropylphenyl)propionaldehyde, 2-methyl-2-(para-isopropylphenyl)propionaldehyde, 2-methyl-3-(para-tert-butylphenyl)propionaldehyde, 2-methyl-3-(tert-butylphenyl)propionaldehyde, methyl 1,6,10-trimethyl-2,5,9-cyclododecatrien-1-yl ketone, methylcedrylone, methyl dihydrojasmonate, methyl β-naphthyl ketone, methyl γ-naphthyl ketone, β-naphthol methyl ether, nerol, para-tert-butylcyclohexyl acetate, tert-butylcyclohexyl acetate, tricyclodecenyl propionate, tricyclodecenyl acetate, 5-(2,2,3-trimethylcyclopent-3-enyl)-3-methylpentan-2-ol, tricyclodecenyl acetate, tricyclodecenyl propionates, phenylethyl alcohol, terpineol, linalool, and 10-undecen-1-al.

8. The system of claim 1 wherein the detector electrodes are is attached to the outer panel.

9. The system of claim 1 wherein the detector electrodes are attached to the absorbent pad.

10. The system of claim 1 wherein the detector circuit is attached to the outer panel.

11. The system of claim 1 wherein the detector circuit is attached to the absorbent pad.

12. The system of claim 1 wherein the power supply is attached to the outer panel.

13. The system of claim 1 wherein the power supply is attached to the absorbent pad.

14. The system of claim 1 wherein the transmitter is attached to the absorbent pad.

15. The system of claim 1 wherein the transmitter is attached to the outer panel.

16. The system of claim 1 wherein the beacon is selected from the group consisting of an computer monitor beacon, electromagnetic speaker beacon, a piezoelectric speaker beacon, a light emitting diode beacon, a liquid crystal diode beacon, and an incandescent lamp beacon.

17. The device of claim 1 wherein the power supply is selected from the group consisting of a battery and a high capacity capacitor.

18. The system of claim 17 wherein the battery is selected from the group consisting of a zinc-carbon battery, zinc-chloride battery, an alkaline/manganese battery, a silver-oxide battery, a lithium battery, a mercury battery, and a water-activated battery.

19. The system of claim 18 wherein the water-activated battery having an anode selected from a magnesium anode, and a magnesium-zinc alloy, the water-activated battery having a cathode selected from the group consisting of silver chloride, cuprous chloride, cuprous bromide, cuprous iodide, and cuprous thiocyanate, copper sulfate, and manganese dioxide.

20. The system of claim 1 wherein the absorbent pad being fabricated of materials selected from the group consisting of fluffed cellulose fibers, textile fibers, web of polymeric fibers, wood pulp fibers, polyester, polypropylene, polyurethane, cellulose sponge, and hydrophilic synthetic sponge.

21. The system of claim 1 wherein the outer panel being selected from the group consisting of polypropylene, polyamide, polyester, polyethylene, ethylene-vinyl acetate, polyurethane, polyolefin blends, co-polyesters, block copolymers and admixtures thereof.

22. The system of claim 1 wherein the absorbent composition being a superabsorbent polymer selected from the group consisting of polyacrylate polymers, starch graft copolymers, cellulose graft copolymers, cross-linked carboxymethylcellulose derivatives, and admixtures thereof.

23. A kit for use in converting a regular disposable diaper into a smart diaper device for use in communication with a remote monitor station, said kit comprising:
at least one pair of detector electrodes;
a detector circuit operatively coupled to the detector electrodes;
a transmitter operatively coupled to the detector circuit, the transmitter configured to transmit an EMF signal; and
a power supply coupled to the detector electrodes.

24. The kit of claim 23 further comprising
a monitor station comprising:
an antenna configured to be responsive to the transmitted EMF signal;
a receiver circuit operatively coupled to the antenna; and
a beacon operatively coupled to the receiver circuit.

25. The kit of claim 24 further comprising a software packet configured to drive the antenna, receiver circuit and beacon when the receiver circuit is operatively interfaced to a personal computer.

26. The kit of claim 23 further comprising a thermocouple operatively coupled to the detector circuit.

27. A method of using a kit to convert a regular disposable diaper into a smart diaper device in communication with a remote monitor station, the method comprising the steps of:
obtaining the kit comprising:
at least one pair of detector electrodes;
a detector circuit operatively coupled to the detector electrodes;
a transmitter operatively coupled to the detector circuit, the transmitter configured to transmit an EMF signal;
a thermocouple operatively coupled to the detector circuit;
a power supply coupled to the detector electrodes, the detector circuit, the thermocouple and the transmitter; and
the monitor station comprising:
an antenna configured to be responsive to the transmitted EMF signal;
a receiver circuit operatively coupled to the antenna; and
a beacon operatively coupled to the receiver circuit; acquiring an unsoiled regular disposable diaper;
inserting the detector electrodes, the detector circuit, the transmitter, the thermocouple and the power supply of the kit into the acquired regular disposable diaper, wherein the steps of inserting results in converting the regular disposable diaper into a smart diaper device;
wearing the smart diaper device;
soiling the smart diaper device;
allowing the detector circuit of the kit to sense an increase in conductivity between the detector electrodes of the kit when the smart diaper device is soiled;
transmitting the EMF signal from the transmitter of the kit when the detector circuit of the kit senses the increase in conductivity between the detector electrodes of the kit;
receiving the transmitted EMF signal with the antenna coupled to the receiver circuit of the monitor station;
activating the beacon of the monitor station in response to receiving the transmitted EMF signal; and
removing the soiled smart diaper device in response to the activated beacon.
